(19) United States
(12) Patent Application Publication
(10) Pub. No.: US 2002/0001597 A1
(43) Pub. Date: Jan. 3, 2002

(54) Chlamydial glycolipid vaccines

(76) Inventors: Elizabeth S. Stuart, Amherst, MA (US); Lloyd H. Semprevivo, Wendall, MA (US); Gary J. Vora, Amherst, MA (US); Erin E. Davis, Amherst, MA (US)

Correspondence Address: J. PETER FASSE, Fish & Richardson P.C., 225 Franklin Street, Boston, MA, 02110-2804, US

(21) Appl. No.: 09/827,490

(22) Filed: Apr. 6, 2001

Related U.S. Application Data
(60) Provisional application No. 60/195,004, filed on Apr. 6, 2000.

(51) Int. Cl. A61K 39/118; C07K 14/195

(52) U.S. Cl. 424/263.1; 530/395

(57) ABSTRACT

Disclosed are compositions for and methods of eliciting in a vertebrate a protective immune response against a member of the genus Chlamydia. The methods include administering to the vertebrate a composition having a carrier group coupled to an oligosaccharide obtained from a chlamydial glycolipid. The composition is administered in an amount sufficient to elicit a protective immune response against the parasite.

### CROSS-REFERENCE TO RELATED APPLICATIONS

This application claims priority from U.S. Provisional Patent application Ser. No. 60/195,004, filed Apr. 6, 2000.

### FIELD OF THE INVENTION

The invention relates to carbohydrate chemistry and vaccinology.

### BACKGROUND OF THE INVENTION

Chlamydia trachomatis and Chlamydia pneumoniae are bacterial pathogens that infect millions of people in both the developed and under-developed regions of the world. When diagnosed, chlamydia (infection with a bacterium of the genus Chlamydia) is treatable and curable with antibiotics. However, as much as 40-80% of women and 10-20% of men who are infected are asymptomatic and susceptible to chronic infections with the bacteria. Such chronic infections can cause or place the asymptomatic patient at high risk for diseases and conditions such as pelvic inflammatory disease, infertility, tubal pregnancy, heart disease, and pneumonia. Thus, a strategy to prevent all chlamydial infections, including asymptomatic ones, would be beneficial.

### SUMMARY OF THE INVENTION

The invention is based on the discovery of an effective chlamydial vaccine based on oligosaccharides derived from one or more chlamydial glycolipids, such as the chlamydial glycolipid exoantigen (GLXA; see, e.g., U.S. Pat. No. 5,840,279). These oligosaccharides, which are cleaved from naturally occurring glycolipids or chemically synthesized, are then covalently linked to a carrier group to form a composition that can be used as a chlamydia vaccine.

Accordingly, the invention features a method of eliciting in a vertebrate a protective immune response (e.g., one including a T cell-dependent antibody response or an antibody response) against a bacterium of the genus Chlamydia by administering to the vertebrate a composition containing a carrier group coupled to an oligosaccharide (or a mixture of oligosaccharides) obtained from a chlamydial glycolipid (i.e., a glycolipid derived from a Chlamydia bacterium). The composition is administered in an amount sufficient to elicit a protective immune response against the bacterium.

The invention further features a composition including a carrier group coupled to an oligosaccharide isolated from a chlamydial glycolipid. The carrier group can be coupled to the oligosaccharide by a linker (e.g., 2-(4-aminophenyl)ethylamine).

Also included is a method of producing a chlamydia vaccine by (1) providing a chlamydial glycolipid, (2) isolating one or more oligosaccharides from the glycolipid, and (3) conjugating the one or more oligosaccharides to a carrier.

In another aspect, the invention features a method of purifying a chlamydial glycolipid by providing an aqueous composition that has been in contact with cells infected with a bacterium of the genus Chlamydia, the aqueous composition containing a chlamydial glycolipid; centrifuging the composition for at least 2 hours (e.g., 3 hours) at 100,000 g or more (e.g., 120,000, 150,000, or 183,000 g) to form a pellet containing the chlamydial glycolipid; and collecting the pellet. This method can include one or more of the following: centrifuging an aqueous mixture at 8000 g or less to produce the aqueous composition, resuspending the pellet in a reaction mixture and digesting the reaction mixture with DNAse, RNAse, and proteinase K to form a digested mixture, and subjecting the digested mixture to affinity chromatography using a monoclonal antibody against GXLA.

The invention also includes a purified GLXA, where the purified GLXA is free of other components as determined by sodium dodecylsulfate gel electrophoreses (SDS-PAGE) and silver staining, using the methods described in Stuart et al., Immunology 68:469-473, 1989. To distinguish whether a band on a SDS-PAGE gel is GLXA, the bands can be transferred to a membrane and visualized as a Western blot using GLXA-specific antibodies. The purified GLXA can be produced by the methods of purifying GLXA described herein.

In some embodiments, the oligosaccharide need not be obtained from a chlamydial glycolipid. Rather, once the structure of the oligosaccharide is known, the oligosaccharide can be chemically, biochemically, or biologically synthesized and purified. In other words, knowing the structure of the oligosaccharides opens the skilled artisan to the opportunity to produce only the immunologically important oligosaccharide portions of the glycolipid in high yields, e.g., by chemical synthesis of the oligosaccharides.

As used herein, “protective immune response” means an immune response capable of reducing or inhibiting, via IgG antibody production or T cell activation, infection by a bacterium of the genus Chlamydia. In the case of a prophylactic composition, the animal or human host has not been infected. Thus, the composition inhibits (partially or completely) any infection or one or more symptoms of infection caused by a subsequent exposure to a bacterium. In the case of a therapeutic composition, the animal or human host exhibits an on-going infection (either symptomatic or asymptomatic), and the composition reduces or inhibits the infection. A protective immune response includes IgG antibody production and T cell activation. A protective composition, e.g., a vaccine, elicits a protective immune response.

A carrier group is a molecule which, when coupled to an oligosaccharide, helps present the oligosaccharide antigen to an immune system. Examples of carrier groups include proteins, such as bovine serum albumin (BSA), tetanus toxoid, CRM 197, and ovalbumin.

An adjuvant is a substance that is incorporated into or is administered simultaneously with the compositions of the invention. Adjuvants increase the duration or level of the immune response in an animal after administration of an antigen. An adjuvant can also facilitate delivery of an antigen into the animal or into specific tissues, cells, or locations throughout the body of the animal. Examples of adjuvants include, but are not limited to, incomplete Freund's, complete Freund's, and alum; and can contain squalene (e.g., MF59, Chiron Corp, Emeryville, Calif.), monophospholipid A (e.g., DetoxJ, Ribi ImmunoChem Research, Inc., Hamilton, Mont.), saponins (QS-21, Cambridge Biotech, Cambridge, Mass.), non-ionic surfactants (NISV, Proteus, Cheshire, United Kingdom), tocols (U.S. Pat. No. 5,667,784), biodegradable-biocompatible poly(D,L-lactide-co-glycolide) (U.S. Pat. No. 5,417,986), immune-stimulating complexes (ISCOMs), and/or liposomes.

A chlamydial glycolipid or oligosaccharides derived from the glycolipid can be present within a composition that can include other components. Some of these components might not be visible on a polyacrylamide gel or Western blot.

The new oligosaccharide compositions of the invention are useful as protective chlamydia vaccines.

Carbohydrates consist of various sugar units, and it is possible to generate antibodies to such sugar units. Therefore anti-idiotypic antibodies, which potentially mimic the carbohydrate subunits, also are included in the invention. The production of high-avidity anti-GLXA monoclonal antibodies is made possible by the methods and compositions of the invention. Thus, different monoclonals can be generated against various epitopes present on the GLXA molecule. In turn, monoclonal antibody (mAb) technology also can be used to make large amounts of anti-idiotype (anti-id) against the complementarity determining regions (CDRs) which define the specificity of a particular V region (idiotype) of an antibody. Monoclonals of proven protective value can be used to generate their respective anti-id monoclonals and these anti-ids then can act as mimics or surrogates for the original carbohydrate.

Protective value of a specific antibody can be established using in vitro incubation procedures in which living pathogens are pre-treated with dilutions of antibody to be tested. These treated organisms then are used to infect monolayers of cells such as HeLa or J774A.1. At 24-48 hours, these monolayers then are fixed and stained with pathogen specific antibody and a secondary antibody conjugated with fluorescein. Microscopic examination of the monolayers using an appropriate UV light source allows detection and quantification of pathogens that have entered the cells and begun replication. A protective antibody will decrease or eliminate pathogen infectivity and/or its survival intracellularly. The protective value of the anti-id monoclonal can be measured using an in vivo system and in this instance is measured by immunization of susceptible hosts with specific monoclonal (e.g., mAb2) followed later by challenge with viable pathogen, such as C. trachomatis serovars, C. pneumoniae, or C. psittaci species. The anti-id can elicit an antibody response (e.g., Ab3, anti-anti-id), and the capacity of this antibody to neutralize the infectivity of the intracellular pathogen would be considered the best evidence of a mAb's therapeutic or prophylactic value (see, e.g., An et al., Pathobiology 65:229-240, 1998). Use of anti-idiotypic antibodies to elicit protective immunity is particularly relevant to those situations in which the native antigen of the particular pathogen is difficult or expensive to obtain.

Genetic vaccines based on the GLXA that can be isolated using the methods of the invention are also part of the invention. Thus, such GLXA or individual sugar moieties of it can be used to generate a series of specific monoclonal antibodies. In turn, as described above, anti-idiotype antibodies to these mAb1's can be generated, and, once an effective anti-idiotype (e.g., mAb2)vaccine for chlamydia is demonstrated, the specific CDRs of the anti-idiotype can be identified, their genetic sequence determined and these sequences utilized to generate what is termed a single chain variable fragment (scFV), which also can serve as an effective immunogen. Appropriate selection and generation of the scFV results in a protein that elicits protection, just as the original mAb2 would do. The gene encoding the unique regions of anti-idiotype can be identified and cloned into a live or non-living vector (e.g., mutant salmonellae or naked DNA). Examples of such vectors are described in Roitt et al., Immunology, 4th ed., Mosby, N.Y., pp. 19.4-19.5, 1996. Materials and procedures for genetic vaccines based on anti-id CDRs are described in Nisenoff et al., Clin. Immunol. Immunopathol. 21:397-403, 1981; Fields et al., Proc. Natl. Acad. Sci. USA 374:739-742, 1995; Westerink et al., Springer Semin Immunopathol. 15:227-234, 1993; Westerink et al., Ann. N.Y. Acad. Sci 730:209-213, 1994; Petitprez et al., Parasitol. Res. 84:38-40, 1998; Tackaberry et al., J. Virol. 67:6815-6819, 1993; Tripathi et al., Mol. Immunol. 35:853-863, 1998; Pantoliano et al., Biochem. 30:10117-10125, 1991; and Hakim et al., J. Immunol. 157:5503-5511, 1996.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

The invention provides new therapeutic and prophylactic compositions for use in treating or inhibiting chlamydial infection. Some pathogenic bacteria (e.g., members of the genus Chlamydia) are not immunogenic, or not sufficiently immunogenic to produce an effective and/or memorable (i.e., long lasting) immune response. The methods of the invention offer a chlamydial antigen presentation strategy that produces a protective or memorable immune response.

### Isolation of Glycolipids from Bacteria

Glycolipids from Chlamydia can be isolated by any method known in the art, or by the methods described below. For example, cells (e.g., McCoy cells [a mouse fibroblast cell line], the mouse macrophage cell line J774A.1, or HeLa 229 cells) can be infected with Chlamydia trachomatis (B serovar) in vitro at an MOI of 10. At 24 hours post-infection 100 U/ml of penicillin are added to increase production of GLXA into the supernatant. GLXA is a chlamydial exoantigen that is secreted into the medium in infected cell cultures and has a molecular weight of about 58 to 62 kDa. At 96 hours post-infection, the GLXA is isolated from the supernatant using standard methods or the methods described in the Example below. Standard methods include hydrophobic gel filtration; treatment with DNAse, RNAse, and proteinase K; solvent extraction; and affinity chromatography (using, e.g., the antibodies described in U.S. Pat. No. 5,840,279). Additional details regarding chlamydial glycolipid isolation can be found in Stuart et al., “Genus glycolipid exoantigen from Chlamydial trachomatis: component preparation, isolation, and analysis,” In: Chlamydial Infection, Oriel et al., eds., 1986, Cambridge University Press, England; Troidle, “Characterization of a genus specific chlamydial antigen,” Ph.D. thesis, 1992, University of Massachusetts, Amherst, Mass.; and Stuart et al., Current Microbiology 28:85-90, 1994.

### Conjugation of Oligosaccharides to Carrier

To produce an antigen useful in a therapeutic or prophylactic composition, such as a chlamydia vaccine, oligosaccharides can be released from an isolated glycolipid. This can be done using, e.g., standard mild acid hydrolysis or glycosidase treatment. See, e.g., Semprevivo et al., Carbohy. Res., 177:222-227, 1988. Additional purification (e.g., by column chromatography) of the oligosaccharides can be performed to isolate oligosaccharides of a specific size range (e.g., 800-3000 daltons). These oligosaccharides can include non-reducing end groups, repeating subunits, and/or core portions of the glycolipid. In addition, the oligosaccharides obtained from a particular glycolipid are expected to contain the same carbohydrate residues as in the glycolipid itself.

The oligosaccharides or mixture of oligosaccharides are then coupled directly or through linkers to a carrier group by conventional methods to form effective immunogens because, as haptens, the oligosaccharides alone are likely to be poor immunogens. Carrier groups can be any polypeptide, organic polymer, or smaller molecule that is suitable for administration to a mammal. When coupled to the oligosaccharides, the carrier groups enhance presentation of oligosaccharide epitopes to a mammalian immune system, thereby inducing an immune response specific for the oligosaccharides and, by extension, for the glycolipid on the surface of the bacterium. The use of a mixture of many different oligosaccharides helps to prevent the target bacterium from adapting and avoiding an immune response.

Any standard chemical linker (e.g., a bi-functional linker containing, for example, reactive amino groups) can be used to couple the oligosaccharides to the carrier group. Examples of such linkers include 1-cyano-4-dimethylaminopyridinium tetrafluoroborate, 4-(4-N-maleimidomethyl)cyclohexane-1-carboxyl hydrazide, and a phenethylamine-isothiocyanate derivative. See, e.g., Lee et al., Vaccine, 14:190-198, 1996; Ragupathi et al., Glycoconjugate J., 15:217-221, 1998; Roy et al., Canad. J. Biochem. Cell Biol., 62:270-275, 1984; and Smith et al., Methods Enzymol., 50:169-171, 1978.

Chemistry and techniques suitable for coupling oligosaccharides to a carrier group such as BSA are known in the art. For example, the carbonyl group of the terminal reducing monosaccharide residue of an oligosaccharide can react with the primary alkylamine group of a linker such as 2-(4-aminophenyl)ethylamine to form an intermediate. This intermediate is then reduced with sodium borohydride to form a stable intermediate and to facilitate a condensation between the terminal arylamino group of the linker portion of the intermediate and a diazo bridge to residues, e.g., lysine residues, of a polypeptide carrier such as BSA. See, e.g., Zopf et al., Meth. Enzymol., 50:163-169, 1978; and Semprevivo et al., supra.

While different oligosaccharide molecules derived from the digestion of a single glycolipid source are coupled to the carrier group using the above methods, oligosaccharides from more than one glycolipid (e.g., glycolipids from two species of Chlamydia or glycolipids of multiple genera of bacteria, only one of which belongs to the genus Chlamydia) also can be linked to a single carrier group. Such multi-specific conjugates are especially useful for the production of broadly protective vaccines or vaccines containing pathogen species-specific antigens. While a vaccine composition produced from GLXA oligosaccharides is expected to protect a vertebrate against all members of the genus Chlamydia, protection against other pathogens (e.g., another pathogenic bacterium or eukaryotic parasite) can be achieved by conjugating oligosaccharide of glycolipids or lipoglycans of these other pathogens to a carrier (either the same or different carrier to which the GLXA oligosaccharides are attached).

### Preparation of Compositions Containing Oligosaccharide/Carrier Group Conjugates

The compositions can include one or more different types of oligosaccharide/carrier group conjugates. For example, conjugates produced from different glycolipids can be mixed together in the same composition to produce a cross-protective vaccine composition. In general, the vaccine compositions can be prophylactic (for uninfected individuals) or therapeutic (for individuals already infected).

The compositions optionally include a pharmaceutically acceptable excipient, such as the diluent phosphate buffered saline or bicarbonate (e.g., 0.24 M NaHCO₃). The excipients used in the new compositions can be chosen by one of ordinary skill in the art, on the basis of the mode and route of administration, and standard pharmaceutical practice, without undue experimentation. Suitable pharmaceutical excipients and diluents, as well as pharmaceutical necessities for their use, are described, e.g., in Remington's Pharmaceutical Sciences. An adjuvant, e.g., a cholera toxin, Escherichia coli heat-labile enterotoxin (LT), liposome, or immune-stimulating complex (ISCOM), can also be included in the vaccine compositions.

To formulate the therapeutic compositions, the oligosaccharide/carrier group conjugates can be further purified by standard methods to remove contaminants such as endotoxins, if present. The final conjugate preparation can be lyophilized and resuspended in sterile, deionized water. Appropriate pharmaceutical excipients can then be added.

The therapeutic compositions can be formulated as a solution, suspension, suppository, tablet, granules, powder, capsules, ointment, or cream. In the preparation of these compositions, at least one pharmaceutical excipient can be included. Examples of pharmaceutical excipients include solvent (e.g., water or physiological saline), solubilizing agent (e.g., ethanol, polysorbates, or Cremophor EL7), agent for achieving isotonicity, preservative, antioxidizing agent, lactose, starch, crystalline cellulose, mannitol, maltose, calcium hydrogen phosphate, light silicic acid anhydride, calcium carbonate, binder (e.g., starch, polyvinylpyrrolidone, hydroxypropyl cellulose, ethyl cellulose, carboxy methyl cellulose, or gum arabic), lubricant (e.g., magnesium stearate, talc, or hardened oils), or stabilizer (e.g., lactose, mannitol, maltose, polysorbates, macrogols, or polyoxyethylene hardened castor oils). If desired, glycerin, dimethylacetamide, 70% sodium lactate, surfactant, or basic substance such as sodium hydroxide, ethylenediamine, ethanolamine, sodium bicarbonate, arginine, meglumine, or trisaminomethane can be added. Biodegradable polymers such as poly-D,L-lactide-co-glycolide or polyglycolide can be used as a bulk matrix if slow release of the composition is desired (see e.g., U.S. Pat. Nos. 5,417,986, 4,675,381, and 4,450,150). Pharmaceutical preparations such as solutions, tablets, granules or capsules can be formed with these components. If the composition is administered orally, flavorings and/or colors can be added.

### Administration of Compositions Containing Oligosaccharide/Carrier Group Conjugates

The new compositions can be administered via any appropriate route, e.g., intravenously, intraarterially, topically, ocularly, by injection, intraperitoneally, intrapleurally, orally, subcutaneously, intramuscularly, sublingually, nasally, by inhalation, intraepidermally, or rectally.

Dosages administered in practicing the invention will depend on factors including the specific vaccine antigen and its concentration in the composition, whether an adjuvant is co-administered with the antigen, the type of adjuvant co-administered, the mode and frequency of administration, and the desired effect (e.g., protection from infection or treatment of an existing infection). Suitable dosages can be determined by one skilled in the art without undue experimentation. In general, the new compositions can be administered in amounts ranging between 0.01 μg and 1 mg of the conjugate per kilogram body weight. If adjuvants are administered with the compositions, amounts of only 1% of the dosages given immediately above can be used. The dosage range for veterinary use can be adjusted according to body weight.

Administration is repeated as necessary, as determined by one skilled in the art. For example, in prophylaxis a priming dose can be followed by three booster doses at weekly intervals. A booster shot can be given at 8 to 12 weeks after the first immunization, and a second booster can be given at 16 to 20 weeks, using the same formulation. Sera or T-cells can be taken from the individual for testing the immune response elicited by the composition against the bacterium (or bacterial surface antigens) in vitro. Methods of assaying antibodies, cytotoxic T-cells, or other mediators of immune function against a specific antigen and assaying their ability to kill or neutralize bacteria in vitro are well known in the art, including the ones described below. See also, e.g., Coligan et al., Current Protocols in Immunology, 1992, Greene Associates Inc. Publishing and John Wiley and Sons, Chapters 2-4 and 6; Crowther, “ELISA Theory and Practice,” Lefkovits I., In: Immunology Methods Manual, Harlow et al., eds.; and Byrne et al., J. Infect. Dis. 168:415-420, 1993. Additional boosters can be given as needed. By varying the amount of the immunogen or composition, the immunization protocol can be optimized for eliciting a maximal immune response.

Before administering the above compositions in humans, toxicity and efficacy testing can be conducted in animals. In an example of efficacy testing, mice can be vaccinated via an oral or parenteral route with a composition containing a oligosaccharide/carrier group conjugate antigen. After the initial vaccination or after optional booster vaccinations, the mice (and corresponding control mice receiving mock vaccinations) are challenged with a dose of pathogenic bacteria. Protective immunity is then determined by an absence or reduction (e.g., a 70%, 80%, 90%, 95%, 99%, or 100% reduction) in the number of viable bacteria in the vaccinated animal (e.g., in a specific tissue) compared to a control animal.

For example, the challenge can be by topical delivery of 2000 TCID₅₀ of K serovar of C. trachomatis onto the vaginal surface of an anesthetized mouse. C. trachomatis serovar K is available as Cat. No. UW-31Cx from the American Type Culture Collection, Manassas, Va. Mice are reclined on their backs after challenge to optimize retention of inoculum during the period of anesthesia. Infections are evaluated by collection of vaginal swabs at weekly intervals for culture and for cytology by direct fluorescent antibody staining for the organism. DFA can be performed using the Syva Direct Reagent (Wampole Laboratories, Wampole, Mass.) according to the manufacturer's protocol. The presence of chlamydial elementary bodies (EB) are graded on a scale of 0 (negative) to 4+ (>10 EB per high power field [hpf]) using an epifluorescence microscope (Carl Zeiss), and compared to control samples. In all cases, slides should be read in a masked fashion without user knowledge of the in vivo treatment associated with each slide.

The level of antibodies that bind to the original glycolipid antigen (e.g., GLXA) in the sera of vaccinated animals can also be evaluated by ELISA. Flat-bottom polyvinylchloride 96-well microtiter plates (Linbro PVC Immunoplate, ICN Biomedicals, Costa Mesa, Calif.) are coated with 50 μl of native glycolipid (e.g., GLXA) in PBS for 1 hour at room temperature. The plates are subsequently blocked with 200 μl of 2% BSA in PBS for 1 hour at room temperature. Each plate is quickly rinsed three times with PBS containing 0.01% Tween 20 and 0.01% sodium azide (PBST-azide) and incubated with 50 μl of animal serum diluted (1:20 to 1:160) in 0.1% BSA/PBS for 1 hour at room temperature. Following the incubation, the plates are washed three times with PBST-azide (3 minutes each). Antibodies that bind to the native antigen are detected by incubation with appropriate, labelled antibodies.

Another indicator of the effectiveness of a vaccine is the increase in the neutralization activity of sera collected from vaccinated animals. For example, sera can be tested for bacteria neutralization on hamster kidney cells following the procedures described in An et al., Pathobiology 65:229-240, 1997; and Su et al., Vaccine 13:1023-1032, 1995; and Byrne et al., supra. Sera are serially diluted (e.g., 1:4 to 1:64) and mixed with an equal volume of purified Chlamydia EB at concentrations known to give about 200 inclusion forming units (IFU) per 5 hpf of microtiter wells. After incubation for 30 minutes at 37° C., 60 μl of the mixture is transferred to flat-bottom microwells containing confluent monolayers of hamster kidney cells. Plates are rocked at 37° C. for 2 hours, after which complete medium containing 1 μg/ml of cycloheximide (Sigma) is added to each well. Plates are incubated in a 5% CO₂ atmosphere for 48-72 hours, fixed with absolute methanol for 5 minutes, and stained with Syva Culture confirmation reagent (Wampole Laboratories, Wampole, Mass.). The stained wells are read on a epifluorescence microscope (Carl Zeiss) at 160× magnification. Results are expressed as the percent reduction of bacteria from sera obtained from unvaccinated control animals compared to sera obtained from vaccinated animals. Any statistically significant reduction of bacteria due to vaccination indicates that the vaccine was effective.

A protective vaccine can also be evaluated by histopathology and by PCR for chlamydial nucleic acid. Anesthetized vaccinated and control animals are sacrificed by exsanguination via the axillary vessels and cervical dislocation. Genital tracts are photographed and documented as to appearance in vivo. The genital tract is then removed under aseptic conditions and photographed ex vivo. One half-tract from each animal is fixed in buffered formalin for paraffin embedding. Paraffin sections are prepared for hematoxylin and eosin staining. Slides remain coded so that human readers are unaware of mouse vaccination status of the samples. Sections are graded for signs of pathology based on a modification of the grading scheme of Rank (Rank et al., Methods in Enzymology 235:83-93, 1994) A reduction in the pathology of vaccinated animal tissue versus control animal tissue indicates that the vaccine was effective.

The other half tract from each animal is divided into upper (L1: ovary, oviduct, and top of uterine horn), mid-tract (L2: mid-uterine horn), and lower tract (L3: lowest portion of uterine horn, cervix, and vagina). These three samples are snap-frozen in liquid nitrogen for extraction of nucleic acids for PCR analysis. Nucleic acids are extracted from genital tract specimens using standard techniques and the primers described in Branigan et al., Arthritis Rheum. 39:1740-1746, 1996; Balin et al., Med. Microbiol. Immunol. 187:23-42, 1998; Gerard et al., Mol. Gen. Genet. 255:637-642, 1997; and Holland et al., Infect. Immun. 60:2040-2047, 1992. Genital tract tissues are treated with proteinase K overnight at 37° C., followed by hot phenol extraction. Following chloroform:ethanol extractions, nucleic acid samples are subjected to PCR, using actin or other control primers as a standard for the reaction. The PCR products are then separated using agarose gel electrophoresis and visualized with ethidium bromide. The presence of a PCR product corresponding to the expected size of a chlamydial target sequence indicates the presence of chlamydial bacteria in a portion of the genital tract. The presence of chlamydial bacteria in unvaccinated control animals and the absence or diminution of chlamydial bacteria in vaccinated control animals indicates that the vaccine was protective.

The amount of bacteria in the genital tract of the test animal can be determined by collecting (e.g., via a swab) shed chlamydial elementary bodies (EBs), the infectious form of the bacteria. The collected EBs can then be quantitated by PCR (e.g. RT-PCR) or by dilution and passage of the EBs into a permissive cell line. Once the EBs are amplified in tissue culture, detection of bacteria can be performed by, e.g., immunofluorescent microscopy to detect bacterial inclusions in host cells.

Alternative animal infection models for Chlamydia include ocular infections. See, e.g., Rank et al., Methods Enzymol. 235:69-83, 1994; Whittum-Hudson et al., Nat. Med. 2:1116-1121, 1996; and Whittum-Hudson et al., Invest. Ophthalmol. Vis. Sci. 36:1976-1987, 1995. In addition, a surrogate marker of chlamydial infection, such as cytokine production, can be monitored. See, e.g., Netea et al., Eur. J. Immunol. 30:541-549, 2000; Kalinin et al., Aviakosm Ekolog Med. 33:48-52, 1999; Vuola et al., Infect. Immun. 68:960-964, 2000; Mavoungou et al., Trop. Med. Int. Health 4:719-727, 1999; and Wang et al., Eur. J. Immunol. 29:3782-3792, 1999.

The dose of the conjugate administered to a subject will depend generally upon the severity of the condition (if any), age, weight, sex, and general health of the subject.

Physicians, pharmacologists, and other skilled artisans are able to determine the most therapeutically effective treatment regimen, which will vary from patient to patient. The potency of a specific composition and its duration of action can require administration on an infrequent basis, including administration in an implant made from a polymer that allows slow release of the conjugate. Skilled artisans are also aware that the treatment regimen must be commensurate with issues of safety and possible toxic effect produced by the conjugate or other components in the compositions, such as adjuvants.

### Variations

The portions of the glycolipid molecule which induce protective antibodies can be determined by raising monoclonal antibodies specific for specific regions of the glycolipid and determining which of these portions of the glycolipid participate in bacteria destruction or elimination. In general, antibodies can be raised by injecting into an animal the immunogenic compositions described herein. Monoclonal antibodies and hybridomas producing them can be cloned and screened (using the original antigen complex as the capture moiety) from a B cell population isolated from the immunized animals using standard methods in the art of molecular biology.

Once antibodies are selected using these screens, the specific oligosaccharide structures to which they bind can be identified by at least two methods. In the first method, the antibodies are used to screen a library of oligosaccharide molecules, each member of the library having a known chemical structure. In the second method, the antibodies are used to “fish out” the specific oligosaccharides from a complex mixture of oligosaccharides produced by digesting a glycolipid using the methods described herein. The structure of the specific oligosaccharides is then identified by chromatographic, spectrometric, or other physical and/or chemical methods known in the art of carbohydrate chemistry.

### EXAMPLE

Isolation of Glycolipid. The chlamydial glycolipid exoantigen, GLXA, was purified from infected cell culture supernatants for the purpose of generating a oligosaccharide conjugate vaccine. Confluent monolayers of HeLa 229 cells were grown in Richter's Improved MEM Insulin (IMEMZO; Irvine Scientific, Santa Ana, Calif.) containing 5% FBS. At the time of infection, the cells were inoculated with Chlamydia trachomatis (K serovar, UW-31/Cx) (VR-887, American Type Culture Collection, Manassas, Va.) at a MOI of 0.1. The IMEMZO was replaced with a complete cycloheximide overlay medium (12-712F; Bio-Whittaker, Walkersville, Md.) containing 10% FBS and 1X L-glutamine. At 96 hours post-infection, the GLXA-containing cell culture supernatant (1.2 L collected from 7200 cm² of confluent HeLa 229 cell monolayers) was collected and centrifuged at 8000×g to remove any cellular debris. The supernatant was then subjected to ultracentrifugation at 183,000×g at 4° C. for 3 hours. Then each pellet was resuspended in 1 ml 0.075 M PBS (i.e., PBS with 0.075 M phosphate) and sequentially digested with DNase (50 μg/ml), RNase (50 μg/ml), and Proteinase K (100 μg/ml) in the presence of 4.2 mM MgCl₂ and 1 mM CaCl₂ (Sigma-Aldrich, St. Louis, Mo.). All digestions were incubated for a minimum of 2 hours at 37° C.

The GLXA-containing solution was then incubated at 85° C. for 2 hours to eliminate any residual Proteinase K activity. The digested material was then centrifuged at 5900×g at 4° C. for 10 minutes to remove any precipitate. The pellets were discarded and the cleared lysate (6 ml) was then subjected to affinity chromatography.

The mouse monoclonal idiotypic antibody, mAb1 (89MS30, described in U.S. Pat. No. 5,840,297) was covalently coupled to 2 ml of rec-Protein A Sepharose 4B beads using the manufacturer's protocol (Zymed, San Francisco, Calif.). The 2 ml of mmAb1-coupled beads and the 6 mL of GLXA-containing cleared supernatant were combined and allowed to incubate at 4° C. for 1 hour with a constant gentle stirring. The column was then poured, and the gel bed was rinsed with 20 gel bed volumes of 0.075 M PBS, followed by 10 gel bed volumes of 1 M NaOH. The gel was again washed with 5 gel bed volumes of 0.075 M PBS. The antigen was eluted from the column using a 0.1 M acetic acid solution, and 1 ml fractions were collected. The GLXA-containing fractions (fractions #1-3) were pooled, yielding 3 ml of the native antigen (antigen #99M100). This antigen was used at a 1:100 dilution in all ELISA based assays as described below. One milliliter of this material was utilized in the protein coupling assay to generate the GLXA oligosaccharide-tetanus toxoid vaccine.

Vaccine Composition. To determine whether oligosaccharides isolated from the glycolipid GLXA might serve as an effective immunogen, the glycolipid was subjected to mild trifluoroacetolysis and to derivatization with 2-(4-aminophenyl)ethylamine as described in Semprevivo, supra. The derivatized intermediate was then coupled to tetanus toxoid (SSI-TetanusTox; Accurate Chemical and Scientific Corp.) to form a oligosaccharide/toxoid conjugate.

To confirm that the resulting conjugate still presents the protective epitope to which anti-GLXA monoclonal antibody 89MS30 (described in U.S. Pat. No. 5,840,297) binds, the conjugate was bound to various amounts of 89MS30 in an ELISA assay. The results indicated that 89MS30 bound to the conjugate in a concentration-dependent manner, indicating that the 89MS30 epitope was still present in the conjugate.

The vaccine composition was completed upon mixing the glycolipid oligosaccharide conjugate with an aluminum hydroxide gel (MAALOX™) in a 1:1 ratio. The aluminum hydroxide gel is an adjuvant suitable for use in humans and is often referred to as alum.

Vaccination of Mice. Four BALB/c mice were injected subcutaneously with 50 μg of the GLXA-tetanus toxoid conjugate on days 0, 10 and 18. Each injection per mouse was 100 μl in volume (50 μl MAALOX™+50 μl vaccine composition).

Evaluating Sera from Vaccinated Mice. All mice, vaccinated and control, were tail bled for serum collection on days—1, 9, 17 and 25. About 50 μl of serum was obtained from each mouse for each time point. The level of antibodies that bind to the original glycolipid antigen (e.g., GLXA) in the sera of vaccinated animals was evaluated by ELISA. Flat-bottom polyvinylchloride 96-well microtiter plates (Linbro PVC Immunoplate, ICN Biomedicals, Costa Mesa, Calif.) were coated with 50 μl of GLXA (99M100) supernatant collected from Chlamydia-infected cultures in PBS for 1 hour at room temperature. The plates were subsequently blocked with 200 μl of 2% BSA in PBS (BSA/PBS) for 1 hour at room temperature. Each plate was quickly rinsed three times with PBS containing 0.01% Tween 20 and 0.01% sodium azide (PBST-azide) and incubated with 50 μl of animal serum diluted (1:20 to 1:160) in 0.1% BSA/PBS for 1 hour at room temperature. Following the incubation, the plates were washed three times with PBST-azide (3 minutes each). The mouse IgG captured by the plate was detected by the addition of 100 μl of goat anti-mouse, alkaline phosphatase-conjugated, polyvalent immunoglobulin (1:2000 dilution; A0162, Sigma-Aldrich, St. Louis, Mo.). The labelled antibody was incubated in the wells for 45 minutes. The wells were washed four times with 200 μl of PBST-azide and once with distilled water. Reactions were then developed with 100 μl paranitrophenyl phosphate substrate (100 mg substrate in 10% diethanolamine, 0.5 mM MgCl₂, and 100 ml distilled water [pH 9.8]). About 1 hour after initiation of the calorimetric reaction, the plate was read on a Vₘₐₓ Kinetic Microplate Reader (Molecular Devices, Sunnyvale, Calif.).

The results of the ELISA assay indicated that all test animals receiving the oligosaccharide/toxoid vaccine produced antibodies that bound to the original GLXA antigen. Thus, the conjugate vaccine is expected to be protective, which can be easily tested using the mouse models of chlamydial infection described herein.

In a separate experiment, serum was obtained from a human patient with a prolonged systemic C. pneumoniae infection. This serum was tested for reactivity against GLXA derived from C. trachomatis in a Western blot. The results indicated that most if not all of the C. trachomatis GLXA bands visible by silver stain were recognized by the patient serum, thereby confirming that (1) chronic active chlamydial infection may be required to expose the GLXA antigen to the immune system, and (2) GLXA is a pan-species antigen.

What is claimed is:

1. A method of eliciting in a vertebrate a protective immune response against a bacterium of the genus Chlamydia, the method comprising administering to the vertebrate a composition comprising a carrier group coupled to an oligosaccharide obtained from a chlamydial glycolipid, the composition being administered in an amount sufficient to elicit a protective immune response against the member.

2. The method of claim 1, wherein the chlamydial glycolipid is glycolipid exoantigen.

3. The method of claim 1, wherein the carrier group is coupled to the oligosaccharide by a linker.

4. The method of claim 3, wherein the linker is 2-(4-aminophenyl)ethylamine.

5. The method of claim 1, wherein the carrier group is coupled to a mixture of oligosaccharides obtained from the glycolipid.

6. The method of claim 5, wherein the mixture of oligosaccharides comprises oligosaccharides having a molecular weight of from 800 to 3000 daltons.

7. A composition comprising a carrier group coupled to an oligosaccharide obtained from a chlamydial glycolipid.

8. The composition of claim 7, wherein the glycolipid is GLXA.

9. The composition of claim 7, wherein the carrier group is coupled to the oligosaccharide by a linker.

10. The composition of claim 9, wherein the linker is 2-(4-aminophenyl)ethylamine.

11. A method of purifying a chlamydial glycolipid, the method comprising providing an aqueous composition that has been in contact with cells infected with a bacterium of the genus Chlamydia, the aqueous composition comprising a chlamydial glycolipid; centrifuging the composition for at least 2 hours at 100,000 g or more to form a pellet comprising the chlamydial glycolipid; and collecting the pellet, thereby purifying the chlamydial glycolipid.

12. The method of claim 11, further comprising centrifuging an aqueous mixture at 8000 g or less to produce the aqueous composition.

13. The method of claim 11, further comprising resuspending the pellet in a reaction mixture and digesting the reaction mixture with DNAse, RNAse, and proteinase K to form a digested mixture.

14. The method of claim 13, further comprising subjecting the digested mixture to affinity chromatography using a monoclonal antibody against chlamydial glycolipid exoantigen.

15. A purified chlamydial glycolipid exoantigen, wherein the purified chlamydial glycolipid exoantigen is free of other components as determined by sodium dodecylsulfate gel electrophoreses and silver staining.

16. A method of eliciting in a vertebrate a protective immune response against a bacterium of the genus Chlamydia, the method comprising administering to the vertebrate a composition comprising a carrier group coupled to an oligosaccharide corresponding to a chlamydial glycolipid, the composition being administered in an amount sufficient to elicit a protective immune response against the member.

17. A composition comprising a carrier group coupled to an oligosaccharide corresponding to a chlamydial glycolipid.
